(19) United States
(12) Patent Application Publication
(10) Pub. No.: US 2007/0128127 A1
(43) Pub. Date: Jun. 7, 2007

(54) Aerosol device containing a silicone, a gradient copolymer and a propellant essentially comprising dimethyl ether

(75) Inventors: Jonathan Gawtrey, Boulogne (FR); Nathalie Mougin, Paris (FR); Isabelle Rollat, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL, Paris (FR)

(21) Appl. No.: 10/568,883

(22) PCT Filed: Aug. 20, 2004
(86) PCT No.: PCT/FR04/02174

§ 371 Date: Dec. 28, 2006

(30) Foreign Application Priority Data
Aug. 22, 2003 (FR) 0310110

(51) Int. Cl. A61K 8/00; A61K 8/89

(52) U.S. Cl. 424/47; 424/70.12

(57) ABSTRACT

The invention relates to an aerosol device containing: a hair treatment composition which comprises, in a cosmetically-acceptable aqueous medium, at least one silicone and at least one compositionally-graded copolymer comprising at least two different monomers and having a mass polydispersity index (Ip) of less than or equal to 2.5; and a propellant essentially comprising dimethylether. The sprayed product can be used, for example, to shape and/or hold styled hair.

The present invention relates to a hair treatment composition contained in an aerosol device, comprising at least one composition-gradient copolymer and at least one silicone, and propelled by a propellant that comprises essentially dimethyl ether, and also to the use of a composition-gradient copolymer in aerosol devices containing a propellant that comprises essentially dimethyl ether.

The haircare products for shaping and/or holding the hairstyle that are the most commonly available on the cosmetics market are spray compositions consisting essentially of a solution, usually an alcoholic solution, and of one or more materials, which are generally polymer resins, known as fixing materials, whose function is to form welds between the hairs, by mixing with various cosmetic adjuvants. The fixing materials are generally fixing polymers, i.e. film-forming polymers that are soluble or dispersible in water or alcohol, such as vinyl acetate/crotonic acid copolymers, anionic or amphoteric acrylic resins, polyurethanes, etc.

For essentially ecological reasons, it is sought to reduce the amount of volatile organic compounds (VOCs) present in the composition. To reduce the amount of VOC and to obtain a low-VOC aerosol device, the organic solvents, for instance ethanol and dimethyl ether, are partially replaced with water.

However, replacing ethanol with water leads to drawbacks such as a degradation of the quality of the spray, blocking of the aerosol device and, occasionally, a loss of cosmetic performance.

The use of particular polymers such as composition-gradient copolymers makes it possible to avoid these drawbacks, but formulation of these polymers alone, for example, in an aqueous medium with a propellant essentially comprising dimethyl ether, leads to bleaching of the hair.

The Applicant has found, surprisingly, that the addition of silicones to an aqueous formulation of composition-gradient copolymers makes it possible to prevent bleaching of the hair and to obtain haircare products for shaping and/or holding the hairstyle that have a low VOC content.

One subject of the present invention is thus an aerosol device containing:
a hair treatment composition comprising, in a cosmetically acceptable aqueous medium, at least one composition-gradient copolymer and at least one silicone as described below; and
a propellant essentially comprising dimethyl ether.

Another subject of the present invention consists of the use of at least one composition-gradient copolymer, in the presence of at least one silicone as described below, in aerosol devices containing a propellant essentially comprising dimethyl ether.

Other characteristics, aspects and advantages of the invention will emerge even more clearly on reading the description and the various examples that follow.

According to the present invention, the hair treatment composition contained in an aerosol device and propelled with a propellant essentially comprising dimethyl ether comprises, in a cosmetically acceptable aqueous medium, at least one silicone as described below and at least one composition-gradient copolymer comprising at least two different monomers, and having a mass polydispersity index (Ip) of less than or equal to 2.5.

The term “propellant essentially comprising dimethyl ether” means a propellant which, besides dimethyl ether, may comprise at least one propellant other than dimethyl ether, chosen from hydrocarbons such as C₁₋₅ alkanes, for example methane, propane, butane or pentane, and compressed gases such as air, nitrogen or carbon dioxide, the dimethyl ether being present in an amount ranging from 5% to 100% by weight, preferably from 20% to 60% by weight and better still from 30% to 50% by weight relative to the total weight of the propellant essentially comprising dimethyl ether.

The amount of propellant(s) other than dimethyl ether is preferably between 0 and 95% by weight, better still between 0 and 60% by weight and even more preferentially between 0 and 40% by weight relative to the total weight of the propellant essentially comprising dimethyl ether. In the case of compressed gases, the amount is preferably between 2 and 14 bar, better still between 4 and 12 bar and even more preferentially between 5 and 11 bar.

The propellant that is particularly preferred consists solely of dimethyl ether.

The propellant essentially comprising dimethyl ether is preferably present in an amount ranging from 20% to 60% by weight, more particularly from 25% to 55% by weight and better still from 30% to 50% by weight relative to the total weight of the hair treatment composition and of the propellant.

The silicones that may be used in accordance with the invention may be soluble or insoluble in the composition. They may in particular be polyorganosiloxanes that are insoluble in the composition of the invention and may be in the form of oils, waxes, resins or gums.

The insoluble silicones are especially dispersed in the compositions in the form of particles generally having a number-average size of between 2 nanometers and 100 micrometers and preferably between 20 nanometers and 20 micrometers (measured with a granulometer).

The organopolysiloxanes are defined in greater detail in Walter Noll's “Chemistry and Technology of Silicones” (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone 7207 by Union Carbide or Silbione 70045 V 2 by Rhodia, decamethylcyclopenta-siloxane sold under the name Volatile Silicone 7158 by Union Carbide, and Silbione 70045 V 5 by Rhodia, and mixtures thereof.

Mention may also be made of cyclocopolymers of the dimethylsiloxane/methylalkylsiloxane type, such as Volatile Silicone FZ 3109 sold by the company Union Carbide, having the chemical structure:

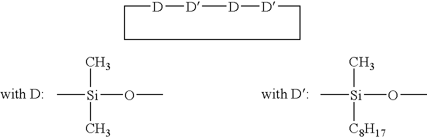

Mention may also be made of mixtures of cyclic silicones with organosilicon compounds, such as the mixture of octamethylcyclo-tetrasiloxane and tetratrimethylsilylpentaerythritol (50/50) and the mixture of octamethylcyclotetrasiloxane and oxy-1,1′-bis-(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Among the non-volatile silicones that may especially be mentioned are polyalkylsiloxanes, polyarylsiloxanes, polyalkylaryl- siloxanes, silicone gums and resins, polyorganosiloxanes modified with organofunctional groups, polysiloxane(A)-polyoxyalkylene(B) linear block copolymers of (A-B)ₙ type with n>3; grafted silicone polymers, with a nonsilicone organic skeleton, consisting of an organic main chain formed from organic monomers not comprising silicone, onto which are grafted, within said chain and also optionally on at least one of its ends, at least one polysiloxane macromonomer; grafted silicone polymers, with a polysiloxane skeleton grafted with nonsilicone organic monomers, comprising a polysiloxane main chain onto which are grafted, within said chain and also optionally on at least one of its ends, at least one organic macromonomer not comprising silicone; and also mixtures thereof.

Examples of polyalkylsiloxanes that may especially be mentioned include polydimethylsiloxanes containing trimethylsilyl end groups with a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a nonlimiting manner, of the following commercial products:
the Silbione oils of the 47 and 70 047 series or the Mirasil oils sold by Rhône-Poulenc, for instance the oil 70 047 V 500 000 ;
the oils of the Mirasil series sold by the company Rhône-Poulenc;
the oils of the 200 series from the company Dow Corning, such as, more particularly, DC200 with a viscosity of 60 000 cSt;
the Viscasil oils from General Electric and certain oils of the SF series (SF 96, SF 18) from General Electric.

Mention may also be made of polydimethylsiloxanes containing dimethylsilanol end groups (Dimethiconol according to the CTFA name) such as the oils of the 48 series from the company Rhône-Poulenc.

In this category of polyalkylsiloxanes, mention may also be made of the products sold under the names “Abil Wax 9800 and 9801” by the company Goldschmidt, which are poly(C₁-C₂₀)alkylsiloxanes.

The polyalkylarylsiloxanes are chosen particularly from linear and/or branched polydimethylmethylphenylsiloxanes or polydimethyl-diphenylsiloxanes, with a viscosity of from 1×10⁻⁵ to 5×10⁻² m²/s at 25° C.

Among these polyalkylarylsiloxanes, mention may be made, by way of example, of the products sold under the following names:
the Silbione oils of the 70 641 series from Rhône-Poulenc;
the oils of the Rhodorsil 70 633 and 763 series from Rhône-Poulenc;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250, SF 1265.

The silicone gums that can be used in accordance with the invention are, in particular, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinylsiloxane.

Silicones that can be used in the composition in accordance with the invention are mixtures such as:
mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 20 gum and 85% SF 96 oil.

The organopolysiloxane resins that can be used in accordance with the invention are crosslinked siloxane systems containing the following units: R₂SiO_{2/2}, R₃SiO_{1/2}, RSiO_{3/2} and Sio_{4/2} in which R represents a hydrocarbon-based group containing 1 to 16 carbon atoms or a phenyl group.

Among these products, those particularly preferred are the ones in which R denotes a C₁-C₄ lower alkyl radical, more particularly methyl, or a phenyl radical.

Among these resins, mention may be made of the product sold under the name “Dow Corning 593” or those sold under the names “Silicone Fluid SS 4230 and SS 4267” by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and containing in their structure one or more organofunctional groups attached via a hydrocarbon-based group.

Among the organomodified silicones, mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;
quaternary ammonium groups, for instance the products sold under the names Abilquat 3272 and Abilquat 3474 by the company Goldschmidt;
thiol groups such as the products sold under the names “GP 72 A” and “GP 71” from Genesee;
alkoxylated groups such as the product sold under the name “Silicone Copolymer F-755” by SWS Silicones and Abil Wax® 2428, 2434 and 2440 by the company Goldschmidt;
hydroxylated groups such as the polyorganosiloxanes containing a hydroxyalkyl function, described in French patent application FR-A-85 16334;
acyloxyalkyl groups such as, for example, the polyorganosiloxanes described in patent U.S. Pat. No. 4,957,732;
anionic groups of carboxylic acid type, such as, for example, in the products described in patent EP 186 507 from the company Chisso Corporation, or of alkylcarboxylic type, such as those present in the product X-22-3701E from the company Shin-Etsu; 2-hydroxyalkyl sulfonate; 2-hydroxyalkyl thiosulfate such as the products sold by the company Goldschmidt under the names “Abil® S201” and “Abil® S255 ”;
hydroxyacylamino groups, such as the polyorganosiloxanes described in patent application EP 342 834. Mention may be made, for example, of the product Q2-8413 from the company Dow Corning.

The silicones that are particularly preferred in the invention are organomodified or non-organomodified polydimethylsiloxanes, such as polyoxyalkylenated polydimethylsiloxanes. Mention may be made especially of those in the form of mixtures of polydimethyl-siloxane /polydimethylsiloxane charged with silica aerogel. By way of example, mention may be made of the product sold under the trade name Dow Corning 1510 EU by the company Dow Corning.

Said silicones are preferably present in an amount ranging from 0.01% to 10% by weight, more particularly from 0.05% to 5% by weight and better still from 0.1% to 2% by weight relative to the total weight of the styling composition and of the propellant.

For the purposes of the present invention, the term “composition-gradient copolymer” means a copolymer in which the distribution of at least one monomer of the polymer chains changes in a given direction along the entire length of these chains and is reproducible from one chain to another.

The composition-gradient copolymers used in the composition according to the invention comprise at least two different monomers, and have a low mass dispersity and also, preferably, a low composition dispersity.

A low mass dispersity means that the chain lengths are approximately identical.

The mass dispersity may be represented with the aid of the mass polydispersity index (Ip) of the copolymer, which is equal to the ratio of the weight-average molecular mass (Mw) to the number-average molecular mass (Mn).

The composition-gradient copolymer used in the invention has a mass polydispersity index of less than or equal to 2.5, preferably between 1.1 and 2.3, better still between 1.15 and 2.0 and more preferentially between 1.2 and 1.9 or 1.8.

The weight-average molecular mass (Mw) of the gradient copolymer is preferably between 5000 and 1 000 000 g/mol, better still between 5500 and 800 000 g/mol and even more preferentially between 6000 and 500 000 g/mol.

Preferably, the number-average molecular mass (Mn) of the composition-gradient copolymer is between 5000 and 1 000 000 g/mol, better still between 5500 and 800 000 g/mol and even more preferentially between 6000 and 500 000 g/mol.

The weight-average (Mw) and number-average (Mn) molecular masses may especially be determined by gel permeation liquid chromatography (GPC) with a refractometric detector and tetrahydrofuran (THF) as eluent, the calibration curve being established with linear polystyrene standards.

The composition-gradient copolymers used in the invention also preferentially have a low composition dispersity. This means that all the copolymer chains have an approximately similar composition (i.e. monomer sequence) and are thus of homogeneous composition.

In order to show that all the copolymer chains have a similar composition, liquid absorption chromatography (LAC) may advantageously be used, allowing the copolymer chains to be separated not according to their molecular weight but according to their polarity. The polarity makes it possible to determine the chemical composition of the polymers constituting the material, since the monomers are known.

Reference may be made to the publication Macromolecules (2001), 34, 2667, which describes the LAC technique.

The composition dispersity may especially be defined from the adsorption chromatography (LAC) curve, which is a curve showing the proportion of polymers as a function of the elution volume. If we take “V^{1/2} min” as the minimum elution volume at the mid-height of the curve, and “V^{1/2} max” as the maximum value of the elution volume at the mid-height of the curve, the composition polydispersity is considered as low if the difference (V^{1/2} max-V^{1/2} min) is less than or equal to 3.5, preferably between 1 and 2.8 and better still between 1.2 and 2.5.

Moreover, the LAC curve has a Gaussian curve profile and more particularly a Gaussian curve profile defined by the formula:

$$y = \frac{A}{w \sqrt{\frac{π}{2}}} \times {ⅇ}^{- 2 \frac{{( x - {x}_{0} )}^{2}}{{w}^{2}}} + {y}_{0 }$$

in which:
x₀ represents the value of x (elution volume) at the centre of the peak,
w is equal to twice the standard deviation of the Gaussian distribution (i.e. 2σ) or corresponds to approximately 0.849 times the width of the peak at mid-height,
A represents the area under the peak,
y₀ represents the value of y corresponding to x₀.

The composition dispersity may also be defined by the value of w as defined above. Preferably, said value w is between 1 and 3, better still between 1.1 and 2.3 and even more preferentially between 1.1 and 2.0.

The gradient copolymers used in the invention may be obtained by living or pseudoliving polymerization.

Living polymerization is a polymerization for which the growth of the polymer chains stops only when the monomer disappears. The number-average mass (Mn) grows as the conversion proceeds. Anionic polymerization is a typical example of living polymerization. Such polymerizations lead to copolymers with a low mass dispersity, i.e. polymers with a mass polydispersity index (Ip) generally of less than 2.

Pseudoliving polymerization is associated with controlled radical polymerization. The main types of controlled radical polymerization that may be mentioned include:
radical polymerization controlled with nitroxides. Reference may be made especially to patent applications WO 96/24620 and WO 00/71501, which describes the tools for this polymerization and their implementation, and also to the articles published by Fischer (Chemical Reviews, 2001, 101, 3581), by Tordo and Gnanou (J. Am. Chem. Soc. 2000, 122, 5929) and by Hawker (J. Am. Chem. Soc. 1999, 121, 3904);
atom transfer radical polymerization, described especially in patent application WO 96/30421 and which proceeds via reversible insertion onto an organometallic complex in a bond of carbon-halogen type;
radical polymerization controlled with sulfur derivatives of xanthate, dithioester, trithiocarbonate or dithiocarbamate type, as described in patent applications FR 2 821 620, WO 98/01478, WO 99/35177, WO 98/58974, WO 99/31144 and WO 97/01478 and in the publication by Rizzardo et al. (Macromolecules, 1998, 31, 5559).

Controlled radical polymerization denotes polymerizations for which the side reactions that usually lead to the disappearance of the propagating species (termination or transfer reaction) are made very unlikely relative to the propagation reaction by means of a free-radical control agent. One drawback of this mode of polymerization lies in the fact that when the concentrations of free radicals become high relative to the monomer concentration, the side reactions once again become a deciding factor and tend to broaden the mass distribution.

By means of these modes of polymerization, the polymer chains of the composition-gradient copolymers used in the invention grow simultaneously and thus incorporate the same ratios of comonomers at each instant. All the chains thus have the same structures or similar structures, resulting in low composition dispersity. These chains also have a low mass polydispersity index.

In the case of random polymers and standard block polymers, the change of the monomers along the polymer chain is no longer gradual and systematic.

As illustrated by the scheme below, a random polymer obtained by standard radical polymerization of two monomers differs from a composition-gradient copolymer by the monomer distribution, which is not identical on all the chains, and by the length of said chains, which is not identical for all the chains.

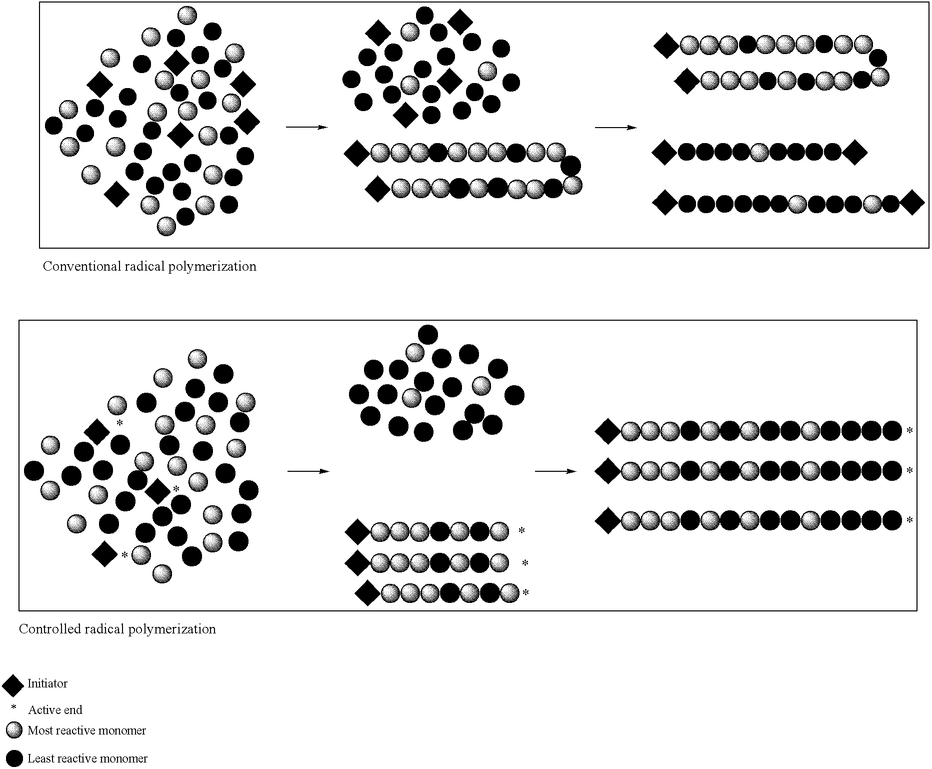

For a theoretical description of composition-gradient copolymers, reference may be made to the following publications:
T. Pakula et al., Macromol. Theory Simul. 5, 987-1006 (1996);
A. Aksimetiev et al. J. of Chem. Physics 111, No. 5;
M. Janco, J. Polym. Sci., Part A: Polym. Chem. (2000), 38(15), 2767-2778;
M. Zaremski et al., Macromolecules (2000), 33(12), 4365-4372;
K. Matyjaszewski et al., J. Phys. Org. Chem. (2000), 13(12), 775-786;
Gray, Polym. Prepr. (Am. Chem. Soc., Div. Polym. Chem.) (2001), 42(2), 337-338;
K. Matyjaszewski, Chem. Rev. (Washington, D.C.) (2001), 101(9), 2921-2990.

Among the composition-gradient copolymers, copolymers with a natural gradient and copolymers with an artificial gradient may be distinguished.

A natural-gradient copolymer is a composition-gradient copolymer which may be obtained by batch synthesis starting with an initial blend of comonomers. The distribution in the chain of the various monomers follows a law deduced from the relative reactivity and from the initial concentrations of monomers. These copolymers constitute the simplest category of composition-gradient copolymers since it is the initial blend that defines the final property of the product.

An artificial-gradient copolymer is a copolymer whose monomer concentration can be varied during the synthesis by means of a process stratagem. In this case, the change from one monomer blend to another in the chain is made by means of a sudden and abrupt change of the monomers in the reaction medium (for example addition of at least one new monomer).

Experimental characterization of the gradient is made by measuring the chemical composition of the polymer during polymerization. This measurement is performed indirectly by determining the change in the concentration of the various monomers at any instant. It may be performed by NMR and UV, for example.

Specifically, for polymers prepared by living or pseudoliving polymerization, the length of the chains is linearly associated with the conversion. By taking a sample of the polymerization solution, at various instants in the polymerization and by measuring the difference in content of each monomer, the composition of the gradient is thus obtained.

In the composition-gradient polymer, the composition distribution of the chains is narrow. In particular, there is no overlap between the chromatographic peak of the composition-gradient copolymer and those of the respective homopolymers. This means that the material obtained as a gradient consists of polymer chains of the same composition, whereas in standard random polymerization, various kinds of chain coexist, including those of the respective homopolymers.

It is possible to characterize the gradient copolymers by means of a vector that is characteristic for each copolymer.

Specifically, given that there are an infinite number of polymers characterized by a given chemical composition, to specify a polymer it is possible to describe the monomer distribution along the chain. This involves a multi-variable description. This vector is one point in space of the chemical compositions.

The exact term is that G is a vector whose coordinates are the concentrations of the monomers along the polymer chain. These concentrations are defined by the rules of the coefficients of reactivity of each of the monomers, and are thus associated with the concentration of the free monomers during the synthesis: as long as the monomer is not in zero concentration in the reaction mixture, it is not in zero concentration in the polymer.

It is thus possible to characterize the composition-gradient copolymers by the function G(x) that defines the composition gradient:
\vec{G}(x)=Σ\vec{[Mi](x)}
in which:
x denotes a normalized position on the polymer chain and
[Mi](x) is the relative concentration, at this position x, of the monomer Mi, expressed in mol %.

The function G(x) thus locally describes the composition of the gradient copolymer.

Two copolymers may have an equivalent overall composition but very different local monomer distributions and thus different gradients.

The factors that determine the gradient are, firstly, the relative coefficients of reactivity of each monomer (referred to as rᵢ for the monomer Mi) which depend mainly on the type of synthetic process used (homogeneous or dispersed) and on the solvents, and, secondly, the initial concentrations of each of the monomers, and also the possible additions of monomers during the polymerization.

The composition-gradient copolymer used in the invention comprises at least two different monomers, which may each be present in a proportion of from 1% to 99% by weight, especially in a proportion of 2-98% by weight and preferably in a proportion of 5-95% by weight relative to the final copolymer.

Preferably, at least one of the monomers of the composition-gradient copolymer is a hydrophilic monomer.

In the present description, the term “hydrophilic monomer” means monomers whose homopolymers are soluble or dispersible in water, or of which an ionic form is.

A homopolymer is said to be water-soluble if it forms a clear solution when it is dissolved at 5% by weight in water, at 25° C.

A homopolymer is said to be water-dispersible if, at 5% by weight in water, at 25° C., it forms a stable suspension of generally spherical fine particles. The mean size of the particles constituting said dispersion is less than 1 μm and more generally ranges between 5 and 400 nm and preferably from 10 to 250 nm. These particle sizes are measured by light scattering.

Preferably, the hydrophilic monomer has a glass transition temperature (referred to as Tg hereinbelow) of greater than or equal to 20° C., better still greater than or equal to 50° C., but may optionally have a Tg of less than or equal to 20° C.

The glass transition temperature (or Tg) may be measured according to ASTM standard D3418-97, by differential calorimetric analysis (or DSC “Differential Scanning Calorimetry”) with a calorimeter over a temperature range of between −100° C. and +150° C., at a heating rate of 10° C./minute in 150 μl aluminum crucibles.

Among the hydrophilic monomers that may be used in the present invention, mention may be made of the following monomers:
C₁-C₄ aminoalkyl (meth)acrylate derivatives and especially N,N-di(C₁-C₄ alkyl)amino(C₁-C₆ alkyl) (meth)acrylates such as N,N-dimethylaminoethyl methacrylate (DMAEMA) or N,N-diethylaminoethyl methacrylate (DEAEMA);
C₁-C₈ dialkylallylamines such as dimethyldiallylamine;
vinylamine;
vinylpyridines and especially 2-vinylpyridine or 4-vinylpyridine;
   and also the mineral-acid or organic-acid salts thereof, or quaternized forms thereof.

Among the mineral acids that may especially be mentioned are sulfuric acid, hydrochloric acid, hydrobromic acid, hydriodic acid, acetic acid, propionic acid, phosphoric acid and boric acid.

Examples of organic acids that may be mentioned include acids comprising one or more carboxylic, sulfonic or phosphonic groups. These may be linear, branched or cyclic aliphatic acids, or alternatively aromatic acids. These acids may also comprise one or more hetero atoms chosen from O and N, for example in the form of hydroxyl groups.

Examples of organic acids that may be mentioned include acids containing an alkyl group such as acetic acid CH₃COOH, polyacids such as terephthalic acid, and hydroxy acids such as citric acid and tartaric acid.

The quaternizing agents may be alkyl halides such as methyl bromide or alkyl sulfates such as methyl sulfate or propane sultone.

Examples of hydrophilic monomers that may also be mentioned include:
ethylenic carboxylic acids, especially monocarboxylic or dicarboxylic acids, containing from 3 to 20 carbon atoms, or salts thereof, such as acrylic acid, methacrylic acid, crotonic acid, itaconic acid, fumaric acid, maleic acid and vinylbenzoic acid;
carboxylic anhydrides bearing a vinyl double bond, containing from 4 to 30 carbon atoms, such as maleic anhydride;
ethylenic sulfonic or phosphonic acids, or salts thereof, such as styrenesulfonic acid, acrylamidopropanesulfonic acid, vinylphosphonic acid and salts thereof, the potassium salt of acryloyloxy-3-sulfopropyl, or the compound of formula CH₂═CHCOOCH₂OCH₂(OH)CH₂SO3⁻Na⁺,
vinyl alcohol.

The neutralizer may be a mineral base, such as LiOH, NaOH, KOH, Ca(OH)₂ or NH₄OH; or an organic base, for example a primary, secondary or tertiary amine, especially an optionally hydroxylated alkylamine, for instance dibutylamine, triethylamine or stearamine, or alternatively 2-amino-2-methylpropanol, monoethanolamine, diethanolamine or stearamidopropyldimethylamine.

Examples of hydrophilic monomers that may also be mentioned include:
unsaturated carboxylic acid amides, for instance acrylamide or methacrylamide, and the N-substituted or N,N-disubstituted amides thereof, such as N-(C₁-C₆ alkyl)(meth)acrylamides, for example N-methylacrylamide, N-isopropylamide, N-butylamide and N-tert-butylamide, and more particularly N-(C₁-C₃ alkyl)(meth)acrylamides, for instance N-methylacrylamide; N,N-di(C₁-C₃ alkyl)(meth)acrylamides, including N,N-dimethylacrylamide; N,N-di(C₁-C₄ alkyl)amino(C₁-C₆ alkyl)(meth)acrylamides, for instance N,N-dimethylaminopropylacrylamide (DMAPA) or N,N-dimethyl-aminopropylmethacrylamide (DMAPMA);
hydroxyalkyl (meth)acrylates, especially those in which the alkyl group contains from 2 to 4 carbon atoms, in particular hydroxyethyl (meth)acrylate;
polyethylene glycol (meth)acrylates (containing from 5 to 100 ethylene oxide or EO units) or glycol (meth)acrylates optionally substituted on the end function thereof with C₁-C₄ alkyl, phosphate, phosphonate or sulfonate groups, for example glyceryl acrylate, methoxypolyethylene glycol (8 or 12 EO) (meth)acrylate; hydroxypolyethylene glycol (meth)acrylate;
C₁-C₄ alkoxyalkyl (meth)acrylates, such as methoxyethyl or ethoxyethyl (meth)acrylate;
polysaccharide (meth)acrylates, for instance sucrose acrylate;
vinylamides such as N-vinylacetamide, which are optionally cyclic, in particular such as vinyllactams such as N-vinylpyrrolidone or N-vinylcaprolactam;
vinyl ethers such as vinyl ethers of an alkyl containing 1 to 12 carbon atoms, such as methyl vinyl ether and ethyl vinyl ether.

Examples of hydrophilic polymers that may also be mentioned include the following compounds of betaine type:
methacrylamidopropoxytrimethylammonium;
N,N-dimethyl-N-methacryloxyethyl-N-(3-sulfopropyl)-ammonium,
3-methacryloylethoxycarbonylpyridinium,
the compound of formula: 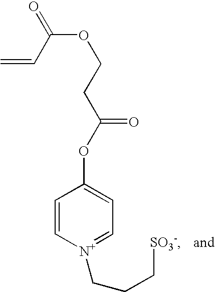
N-(3-sulfopropyl)-4-vinylpyridinium of formula:

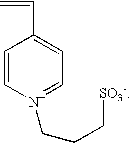

At least one of the monomers of the composition-gradient copolymer may also be a hydrophobic monomer, in particular a hydrophobic monomer that may be made hydrophilic after polymerization, or a blend of such monomers. The hydrophobic monomer(s) may be made hydrophilic, for example, by chemical reaction, especially by hydrolysis, or by chemical modification, in particular of an ester function via incorporation of chains comprising a hydrophilic unit, for example, of carboxylic acid type.

Preferably, the hydrophilic monomers are chosen from N,N-dimethylaminoethyl methacrylate (DMAEMA), acrylic acid, methacrylic acid, crotonic acid, styrenesulfonic acid, acrylamido-propanesulfonic acid, dimethylaminopropylmethacrylamide (DMAPMA); styrene-sulfonate, hydroxyethyl acrylate, glyceryl acrylate, methoxyethyl (meth)acrylate, ethoxyethyl (meth)acrylate, methoxypolyethylene glycol (8 or 12 EO) (meth)acrylate; hydroxy-polyethylene glycol (meth)acrylate; N-vinylpyrrolidone, N-vinyl-caprolactam, acrylamide and N,N-dimethylacrylamide.

The hydrophilic monomer(s) may be present in a proportion of from 1% to 99% by weight, preferably from 2% to 70% by weight, better still from 5% to 50% by weight and even more preferentially from 10% to 30% by weight relative to the total weight of the copolymer.

At least one of the monomers of the composition-gradient copolymer used in the invention may preferably be a hydrophobic monomer.

Among the hydrophobic monomers that may be used in the present invention, mention may be made of:
ethylenic hydrocarbons containing from 2 to 30 carbon atoms, such as ethylene, isoprene and butadiene;
the acrylates of formula CH₂═CHCOOR₁, in which R₁ represents a linear, branched or cyclic, saturated or unsaturated hydrocarbon-based group containing from 1 to 30 carbon atoms, in which is(are) optionally intercalated one or more hetero atoms chosen from O, N, S and Si, said hydrocarbon-based group also possibly being optionally substituted with one or more substituents chosen from hydroxyl groups and halogen atoms (Cl, Br, I and F).

As examples of hydrocarbon-based groups for R₁, mention may be made especially of a C₁-C₃₀ alkyl group, said alkyl group also possibly being optionally substituted with one or more substituents comprising Si; a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; said cycloalkyl, aryl, aralkyl and heterocyclic groups also possibly being optionally substituted with one or more linear or branched alkyl groups of 1 to 4 carbon atoms, in which is(are) optionally intercalated one or more hetero atoms chosen from O, N, S and P, said alkyl groups also possibly being optionally substituted with one or more substituents chosen from hydroxyl groups and halogen atoms (Cl, Br, I and F) or Si.

As preferred examples of such groups R₁, mention may be made especially of methyl, ethyl, propyl, butyl, isobutyl, tert-butyl, hexyl, ethylhexyl, octyl, lauryl, isooctyl, isodecyl, t-butylcyclohexyl, t-butylbenzyl, furfuryl, isobornyl, ethylperfluorooctyl and propyl-polydimethylsiloxane groups.

R₁ may also be a group —(R₁₀)—(OC₂H₄)ₙ—OR₁₁, with x=0 or 1, R₁₀=saturated or unsaturated divalent hydrocarbon-based group such as linear or branched alkylene or alkenylene containing from 1 to 30 carbon atoms, n=5 to 100 and R₁₁=H or CH₃; and especially a methoxy-(PEO)₈-stearyl group with PEO=poly(ethylene oxide);
the methacrylates of formula: CH₂═C(CH₃)—COOR₂, in which R₂ represents a saturated or unsaturated, linear, branched or cyclic hydrocarbon-based group containing from 1 to 30 carbon atoms, in which is(are) optionally intercalated one or more hetero atoms chosen from O, N, S and Si, said hydrocarbon-based group also possibly being optionally substituted with one or more substituents chosen from hydroxyl groups and halogen atoms (Cl, Br, I or F).

As examples of hydrocarbon-based groups for R₂, mention may be made especially of a linear or branched alkyl group of 1 to 30 carbon atoms, said alkyl group also possibly being optionally substituted with one or more substituents comprising Si; a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; said cycloalkyl, aryl, aralkyl and heterocyclic groups also possibly being optionally substituted with one or more linear or branched alkyl groups of 1 to 4 carbon atoms, in which is(are) optionally intercalated one or more hetero atoms chosen from O, N, S and P, said alkyl groups also possibly being optionally substituted with one or more substituents chosen from hydroxyl groups and halogen atoms (Cl, Br, I and F).

Preferred examples of groups R₂ are methyl, ethyl, propyl, n-butyl, isobutyl, hexyl, ethylhexyl, octyl, lauryl, isooctyl, isodecyl, dodecyl, tert-butylcyclohexyl, isobornyl, tert-butylbenzyl, ethylperfluorooctyl and propylpolydimethylsiloxane; R₂ may also be a group —(R₁₀)ₓ—(OC₂H₄)ₙ—OR₁₁, with x=0 or 1, R₁₀=saturated or unsaturated, linear or branched divalent hydrocarbon-based group, such as alkylene or alkenylene containing from 1 to 30 carbon atoms, n=5 to 100 and R₁₁=H or CH₃; and especially a methoxy-(PEO)₈-stearyl group.

Examples of methacrylate monomers are methyl, ethyl, n-butyl, isobutyl, t-butylcyclohexyl, t-butylbenzyl and isobornyl methacrylate;
N—or N,N-substituted unsaturated carboxylic acid amides such as N—(C₈-₃₀ alkyl)(meth)acrylamides, for instance N-octyl-acrylamide;
the vinyl esters of formula: R₃—CO—O—CH═CH₂ in which R₃ represents a linear or branched alkyl group of 2 to 30 carbon atoms, especially vinyl propionate, vinyl butyrate, vinyl ethylhexanoate, vinyl neononanoate and vinyl neododecanoate;
the vinyl compounds of formula: CH₂═CH—R₄, in which R₄ is a —OC(O)—CH₃ group or a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; said cycloalkyl, aryl, aralkyl and heterocyclic groups possibly being optionally substituted with one or more substituents chosen from hydroxyl groups, halogen atoms and linear or branched alkyl groups of 1 to 4 carbon atoms, in which is(are) optionally intercalated one or more hetero atoms chosen from O, N, S and P, and said alkyl groups also possibly being optionally substituted with one or more substituents chosen from hydroxyl groups and halogen atoms (Cl, Br, I and F) or Si.

Examples of such vinyl monomers are vinylcyclohexane, styrene and vinyl acetate.

Preferably, the hydrophobic monomers are chosen from:
isoprene and butadiene;
methyl, ethyl, isobutyl, n-butyl, tert-butyl, ethylhexyl, furfuryl, isobornyl, tert-butylcyclohexyl or tert-butylbenzyl acrylate;
methyl, ethyl, n-butyl, isobutyl, hexyl or ethylhexyl methacrylate;
N—(C₈₋₁₂ alkyl)(meth)acrylamides such as N-octylacrylamide;
the vinyl esters of formula: R₃—CO—O—CH═CH₂ in which R₃ represents a linear or branched alkyl group of 6 to 30 carbon atoms, especially vinyl neononanoate and vinyl neododecanoate;
styrene;
vinyl acetate and vinylcyclohexane.

These monomers may be present in a proportion of from 1% to 99% by weight, preferably from 10% to 90% by weight, better still from 20% to 80% by weight and even more preferentially from 25% to 75% by weight relative to the total weight of the copolymer.

In one preferred embodiment, the composition-gradient copolymer used in the invention comprises three different monomers that may be present in a proportion of 5-90% by weight each and preferably 7-86% by weight each relative to the total weight of the copolymer.

In particular, the copolymer may comprise 5-25% by weight of a first monomer, 5-25% by weight of a second monomer and 50-90% by weight of a third monomer.

Preferentially, the copolymer according to the invention may comprise 5-25% by weight of a hydrophilic monomer, 50-90% by weight of a monomer with a Tg of less than or equal to 20° C. and 5-25% by weight of an additional monomer.

A person skilled in the art will know how to select the monomers and the amounts thereof as a function of the desired result, on the basis of his general knowledge, especially on the relative reactivity of each monomer.

Thus, if a copolymer containing hydrophilic units in the core of a polymer chain is desired, a difunctional initiator and a monomer blend such that the reactivity of the hydrophilic monomers is greater than that of the other monomers will preferably be selected.

Moreover, it has been found that the preparation processes used make it possible to adjust and to modify the Tg value(s) of the copolymer, and thus to obtain a composition-gradient copolymer having one or more given Tg value(s).

The composition-gradient copolymers used in the invention may be prepared by a person skilled in the art according to the following procedure:

1/A blend of the various monomers is prepared, optionally in a solvent, preferably in a reactor and with stirring. A radical polymerization initiator and a polymerization control agent are added. The mixture is preferably placed under an atmosphere of a gas that is inert relative to a radical polymerization, such as nitrogen or argon.

Optional polymerization solvents that may be chosen include alkyl acetates such as butyl acetate or ethyl acetate, aromatic solvents such as toluene, ketonic solvents such as methyl ethyl ketone, or alcohols such as ethanol. When the monomer blend is water-miscible, it may be advantageously used as solvent or co-solvent.

2/The mixture is brought to the desired polymerization temperature with stirring. This temperature is preferably chosen within a range from 10° C. to 160° C. and more preferentially from 25° C. to 130° C.

The choice of the polymerization temperature is preferably optimized as a function of the chemical composition of the monomer blend. Thus, monomers having very high propagation kinetic constants and a lower affinity for the control agent will preferably be polymerized at low temperature (for example, in the case of a large proportion of methacrylic derivatives, a polymerization at a temperature of between 25° C. and 80° C. will be preferred). 3/The polymerization medium is optionally modified during the polymerization, before reaching 90% conversion of the initial monomers, by supplemental addition of one or more monomers, especially of the initial blend. This addition may be performed in various ways, which may range from sudden addition in a single portion to continuous addition over the entire duration of the polymerization. 4/The polymerization is stopped when the desired degree of conversion is reached. The overall composition of the copolymer depends on this conversion. Preferably, the polymerization is stopped after having reached at least 50% conversion, especially at least 60% and preferentially after having reached at least 90% conversion. 5/The possible residual monomers may be removed by any known method, such as by evaporation or by adding an amount of standard polymerization initiator such as peroxide or azo derivatives.

In a first embodiment, the polymerization control agent that may be used is a nitroxide of formula (I), alone or as a mixture: 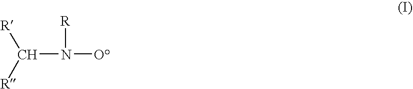
in which:
R and R′, independently of each other, are linear or branched saturated hydrocarbon-based (alkyl) groups containing 1 to 40 carbon atoms, optionally substituted with one or more groups chosen from —OR′₄, —COOR′₄ and —NHR′₄ (with R′₄ representing H or a linear or branched saturated hydrocarbon-based (alkyl) group containing 1 to 40 carbon atoms), R and R′ also possibly being connected so as to form a ring.

In particular, R and R′ are linear or branched alkyl groups containing from 1 to 12 carbon atoms, especially methyl, ethyl, propyl, n-butyl, isobutyl, tert-butyl or pentyl groups. Preferably, R and R′ are both tert-butyl groups.
R″ is a monovalent group with a molar mass (Mp) of greater than 16 g/mol, especially a phosphorus-containing group of formula: 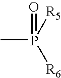
   in which R₅ and R₆, independently of each other, are linear or branched saturated hydrocarbon-based groups, preferably alkyl, containing 1 to 40 carbon atoms, optionally substituted with one or more groups chosen from —OR₄, —COOR′₄ and —NHR′₄ (with R′₄ representing H or a saturated linear or branched hydrocarbon-based group, preferably alkyl, containing 1 to 40 carbon atoms), R₅ and R₆ also possibly being connected so as to form a ring.

In particular, R₅ and R₆ are linear or branched alkyl groups containing 1 to 12 carbon atoms, especially methyl, ethyl, propyl, n-butyl, isobutyl, tert-butyl or pentyl groups. Preferably, R₅ and R₆ are both ethyl groups.

The radical polymerization initiator may be chosen from any common polymerization initiators, such as compounds of azo type and especially azobis(isobutyronitrile), or of peroxide type such as organic peroxides containing 6-30 carbon atoms, especially benzoyl peroxide.

Preferably, a nitroxide/initiator mole ratio of between 1 and 2.5 is respected; this ratio may be between 2 and 2.5 when it is considered that one mole of initiator gives rise to two moles of polymer chains, and may be between 1 and 1.25 for monofunctional initiators.

In a second particular embodiment, alkoxyamines of formula (II) may be used as radical polymerization initiator, and may be advantageously chosen to initiate the polymerization and simultaneously release the nitroxide controlling this polymerization. 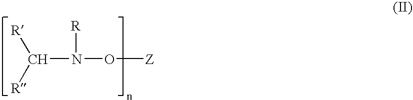
in which:
R, R′ and R″ have the meanings given above,
n is an integer less than or equal to 8 and preferably between 1 and 3 ;
Z is a monovalent or multivalent radical, especially a styryl, acryl or methacryl radical.

A nitroxide of formula (I) may also be added to the alkoxyamine of formula (II), in a proportion ranging from 0 to 20 mol % relative to the number of moles of alkoxyamine functions (one mole of multivalent alkoxyamine give a number of alkoxyamine functions proportional to its valency) so as to improve the quality of the polymerization control.

A person skilled in the art will know how to select the initiator as a function of the needs of the application. Thus, a monofunctional initiator will lead to dissymmetric chains, whereas a polyfunctional initiator will lead to macromolecules having symmetry from a core.

The copolymers may be present in the composition in dissolved form, for example in water or an organic solvent, or alternatively in the form of an aqueous or organic dispersion.

It is possible to prepare an aqueous solution of the copolymer directly by mixing the polymer with water, optionally with heating.

The polymer may also be dissolved in an organic solvent with a boiling point lower than that of water (for example acetone or methyl ethyl ketone), to a solids content of between 20% and 90% by weight.

When the hydrophilic monomers are of acid type, a solution preferably of at least 1M of base, such as a hydroxonium ion (OH⁻) salt, an amine (ammonia), a carbonate (CO₃²⁻) or hydrogen carbonate (HCO₃⁻) salt or an organic neutralizer may be added to the organic solution. In the case of hydrophilic monomers of amine type, a solution, preferably at least 1M, of acid may be added. The water is then added to the solution with vigorous stirring in a proportion such that the solids content obtained is between 1% and 80% by weight. Optionally, the water may be replaced with an aqueous-alcoholic mixture, in proportions ranging from 99/1 to 50/50. The solvent is evaporated off by stirring the solution at 100° C. The concentration is continued until the desired solids content is obtained.

The composition-gradient copolymer used in the context of the present invention is generally present in an amount ranging from 0.1% to 20% by weight, preferably ranging from 1% to 17% by weight and better still ranging from 5% to 15% by weight relative to the total weight of the hair treatment composition and of the propellant.

The term “cosmetically acceptable medium” means any medium that is compatible with keratin materials and especially with the hair.

The cosmetically acceptable medium comprises water and/or one or more cosmetically acceptable solvents. This(these) cosmetically acceptable solvent(s) is or are especially chosen from C₁-C₄ lower alcohols, for instance ethanol, isopropanol, tert-butanol or n-butanol; polyols, for instance propylene glycol, and polyol ethers; acetone; and mixtures thereof, the solvent that is particularly preferred being a C₁-C₄ lower alcohol and even more particularly ethanol.

The proportion of water may be between 20% and 95% by weight and preferably between 25% and 90% by weight relative to the total weight of the hair treatment composition and of the propellant. Advantageously, the medium is aqueous or an aqueous-alcoholic mixture. When the alcohol is present, its proportion is especially between 1% and 70% by weight, preferably between 5% and 65% by weight and even more preferentially between 10% and 60% by weight relative to the total weight of the hair treatment composition and of the propellant.

The hair treatment composition according to the invention may also contain at least one adjuvant chosen from silicones in soluble, dispersed or microdispersed form, other than those described above, nonionic, anionic, cationic and amphoteric surfactants, ceramides and pseudoceramides, vitamins and provitamins including panthenol, plant, animal, mineral and synthetic oils, waxes, ceramides and pseudoceramides, water-soluble and liposoluble, silicone or nonsilicone sunscreens, mineral and organic, colored or uncolored pigments, dyes, nacreous agents and opacifiers, sequestrants, plasticizers, solubilizers, acidifying agents, basifying agents, mineral and organic thickeners, antioxidants, hydroxy acids, penetrants, fragrances and preserving agents.

A person skilled in the art will take care to select the optional additives and the amount thereof such that they do not harm the properties of the compositions of the present invention.

These additives are present in the composition according to the invention in an amount ranging from 0 to 20% by weight relative to the total weight of the composition.

The hair treatment compositions contained in the device according to the invention may be used for shaping and/or holding the hairstyle, for example as fixing and/or hold compositions for the hair, haircare compositions, shampoos, hair conditioning compositions, such as compositions for giving the hair softness, or alternatively hair makeup compositions.

More particularly, the present invention also relates to the use of the product vaporized by the aerosol device according to the invention, as a hair lacquer.

The present invention also relates to a styling process, comprising the step consisting in vaporizing the hair treatment composition contained in the aerosol device according to the invention onto wet or dry hair.

The examples that follow are given as illustrations of the present invention. All the amounts indicated are expressed as weight percentages relative to the total weight of the composition and of the propellant, unless otherwise indicated.

### EXAMPLES

### Example 1

The composition below was prepared by mixing together the ingredients indicated below.

| | Amount |
|---|---|
| Gradient copolymer: poly(methacrylic acid/styrene)poly(butyl | 15 AM |
| acrylate) | |
| Silicone sold under the trade name Dow Corning 1510 EU | 0.1 AM |
| Water | 49.9 |
| DME | 35 |

AM: active material

When applied to the hair, the composition allows shaping and hold of the hairstyle to be obtained without bleaching the hair.

### Example 2

The composition below was prepared by mixing together the ingredients indicated below.

| | Amount |
|---|---|
| Gadient copolymer: poly(methacrylic | 15 AM |
| acid/styrene)poly(ethyl acrylate) | |
| Silicone sold under the trade name Dow Corning | 0.1 AM |
| 1510 EU | |
| Water | 49.9 |
| DME | 35 |

AM: active material

When applied to the hair, the composition allows shaping and hold of the hairstyle to be obtained without bleaching the hair.

### Example 3

The composition below was prepared by mixing together the ingredients indicated below.

| | Amount |
|---|---|
| Gadient copolymer: poly(methacrylic | 15 AM |
| acid/styrene)poly(methyl acrylate/butyl acrylate) | |
| Silicone sold under the trade name Dow Corning | 0.1 AM |
| 11510 EU | |
| Water | 49.9 |
| DME | 35 |

AM: active material

When applied to the hair, the composition allows shaping and hold of the hairstyle to be obtained without bleaching the hair.

### Example 4

The composition below was prepared by mixing together the ingredients indicated below.

| | Amount |
|---|---|
| Gradient copolymer: methacrylic acid/styrene/ | 5 AM |
| butyl acrylate* | |
| Silicone sold under the trade name Dow Corning | 0.1 AM |
| 1510 EU | |
| Water | 39.9 |
| Ethanol | 20 |
| Dimethyl ether | 35 |

AM: active material
*: 100% neutralized with aminomethylpropanol (AMP)

When applied to the hair, the composition allows shaping and hold of the hairstyle to be obtained without bleaching the hair.

### Example 5

The composition below was prepared by mixing together the ingredients indicated below.

| | Amount |
|---|---|
| Gradient copolymer: methacrylic acid/styrene/butyl acrylate* | 5 AM |
| Silicone sold under the trade name Dow Corning 1510 EU | 0.1 AM |
| Water | 14.9 |
| Ethanol | 45 |
| Dimethyl ether | 35 |

AM: active material
*100% neutralized with aminomethylpropanol (AMP)

When applied to the hair, the composition allows shaping and hold of the hairstyle to be obtained without bleaching the hair.

1. An aerosol device containing:
a hair treatment composition comprising, in a cosmetically acceptable aqueous medium, at least one silicone and at least one composition-gradient copolymer comprising at least two different monomers, and having a mass polydispersity index (Ip) of less than or equal to 2.5, and
a propellant essentially comprising dimethyl ether.

2. The aerosol device as claimed in claim 1, characterized in that the propellant essentially comprising dimethyl ether comprises from 5% to 100% by weight and preferably from 20% to 60% by weight of dimethyl ether relative to the total weight of the propellant.

3. The aerosol device as claimed in claim 2, characterized in that the propellant other than dimethyl ether is chosen from hydrocarbons and compressed gases.

4. The aerosol device as claimed in claim 3, characterized in that the propellant other than dimethyl ether is chosen from C₁₋₅ alkanes, air, nitrogen and carbon dioxide.

5. The aerosol device as claimed in claim 3 or 4, characterized in that the propellant other than dimethyl ether is contained in an amount of from 0 to 95% by weight and preferably from 0 to 60% by weight relative to the total weight of the propellant.

6. The aerosol device as claimed in claim 3, 4 or 5, characterized in that the pressure of the compressed gas is between 2 and 14 bar.

7. The aerosol device as claimed in claim 1, characterized in that the propellant consists solely of dimethyl ether.

8. The aerosol device as claimed in any one of the preceding claims, characterized in that the mass polydispersity index (Ip) is between 1.1 and 2.3.

9. The aerosol device as claimed in any one of the preceding claims, characterized in that the silicone(s) is(are) chosen from organomodified or non-organomodified polydimethylsiloxanes.

10. The aerosol device as claimed in any one of the preceding claims, characterized in that the silicone(s) is(are) present in an amount ranging from 0.01% to 10% by weight relative to the total weight of the hair treatment composition and of the propellant.

11. The aerosol device as claimed in any one of the preceding claims, characterized in that the weight-average molecular mass of the composition-gradient copolymer is between 5000 and 1 000 000 g/mol.

12. The aerosol device as claimed in any one of the preceding claims, characterized in that the number-average molecular mass of the composition-gradient copolymer is between 5000 and 1 000 000 g/mol.

13. The aerosol device as claimed in any one of the preceding claims, characterized in that the composition-gradient copolymer is such that, on the adsorption chromatography (LAC) curve representing the proportion of polymers as a function of the elution volume, the difference (V^{1/2} max-V^{1/2} min) is less than or equal to 3.5 and preferably between 1 and 2.8, “V^{1/2} min” being the minimum value of the elution volume at the mid-height of the curve, and “V^{1/2} max” being the maximum value of the elution volume at the mid-height of the curve.

14. The aerosol device as claimed in any one of the preceding claims, characterized in that at least one of the monomers of the composition-gradient copolymer is a hydrophilic monomer.

15. The aerosol device as claimed in claim 14, characterized in that the hydrophilic monomer is chosen from:
C₁-C₄ aminoalkyl (meth)acrylate derivatives;
C₁-C₈ dialkylallylamines;
vinylamine;
vinylpyridines;
and also the mineral acid or organic acid salts thereof, or the quaternized forms thereof;
ethylenic carboxylic acids containing from 3 to 20 carbon atoms, or salts thereof;
carboxylic anhydrides bearing a vinyl double bond, containing from 4 to 30 carbon atoms;
ethylenic sulfonic or phosphonic acids, and salts thereof;
vinyl alcohol;
acrylamide or methacrylamide, N—(C₁-C₆ alkyl)(meth)acrylamides, N,N-di(C₁-C₃ alkyl)(meth)acrylamides, N,N-di(C₁-C₄ alkyl)amino(C₁-C₆ alkyl)(meth)acrylamides;
hydroxy (C₂-C₄ alkyl) (meth)acrylates;
polyethylene glycol (5 to 100 ethylene oxide units) (meth)acrylates or glycol (meth)acrylates, which are unsubstituted or substituted on their end function with C₁₋₄ alkyl, phosphate, phosphonate or sulfonate groups;
C₁₋₄ alkoxyalkyl (meth)acrylates;
polysaccharide (meth)acrylates;
optionally cyclic vinylamides;
vinyl ethers;
methacrylamidopropoxytrimethylammonium;
N,N-dimethyl-N-methacryloxyethyl-N-(3-sulfopropyl)ammonium,
3-methacryloylethoxycarbonylpyridinium,
the compound of formula: 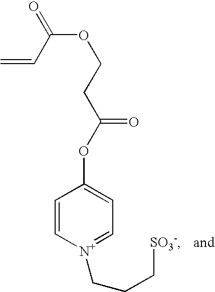
N-(3-sulfopropyl)-4-vinylpyridinium of formula:

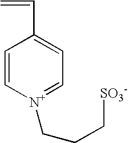

16. The aerosol device as claimed in claim 15, characterized in that the hydrophilic monomer is chosen from N,N-dimethylaminoethyl methacrylate (DMAEMA), acrylic acid, methacrylic acid, crotonic acid, styrenesulfonic acid, acrylamido-propanesulfonic acid, dimethylaminopropylmethacrylamide (DMAPMA); styrene-sulfonate, hydroxyethyl acrylate, glyceryl acrylate, methoxyethyl (meth)acrylate, ethoxyethyl (meth)acrylate, methoxypolyethylene glycol (8 or 12 EO) (meth)acrylate; hydroxy-polyethylene glycol (meth)acrylate; N-vinylpyrrolidone, N-vinyl-caprolactam, acrylamide and N,N-dimethylacrylamide.

17. The aerosol device as claimed in any one of the preceding claims, characterized in that at least one of the monomers of the composition-gradient copolymer is a hydrophobic monomer.

18. The aerosol device as claimed in claim 17, characterized in that the hydrophobic monomer is chosen from:
ethylenic hydrocarbons containing from 2 to 30 carbon atoms;
the acrylates of formula CH₂═CHCOOR₁, in which R₁represents a saturated or unsaturated, linear, branched or cyclic hydrocarbon-based group containing from 1 to 30 carbon atoms, in which is(are) optionally intercalated one or more hetero atoms chosen from O, N, S and Si, said hydrocarbon-based group also possibly being substituted with one or more substituents chosen from hydroxyl groups and halogen atoms,
or R₁ represents a group —(R₁₀)ₓ—(OC₂H₄)_{n—OR}₁₁, with x=0 or 1, R₁₀=saturated or unsaturated, linear or branched divalent hydrocarbon-based group containing from 1 to 30 carbon atoms, n=5 to 100 and R₁₁=H or CH₃;
the methacrylates of formula: CH₂═C(CH₃)—COOR₂, in which R₂ represents a saturated or unsaturated, linear, branched or cyclic hydrocarbon-based group containing from 1 to 30 carbon atoms, in which is(are) optionally intercalated one or more hetero atoms chosen from O, N, S and Si, said hydrocarbon-based group also being optionally substituted with one or more substituents chosen from hydroxyl groups and halogen atoms,
or R₂ represents —(R₁₀)ₓ—(OC₂H₄)ₙ—OR₁₁, with x=0 or 1, R₁₀=saturated or unsaturated, linear or branched divalent hydrocarbon-based group containing from 1 to 30 carbon atoms, n=5 to 100 and R₁₁=H or CH₃;
N—(C₈₋₃₀ alkyl)(meth)acrylamides;
the vinyl esters of formula: R₃—CO—O—CH═CH₂ in which R₃ represents a linear or branched alkyl group of 2 to 12 carbon atoms;
the vinyl compounds of formula: CH₂═CH—R₄ in which R₄ is a group —OC(O)—CH₃, a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; said cycloalkyl, aryl, aralkyl and heterocyclic groups being optionally substituted with one or more substituents chosen from hydroxyl groups, halogen atoms and linear or branched alkyl groups of 1 to 4 carbon atoms, in which is(are) optionally intercalated one or more hetero atoms chosen from O, N, S and P, and said alkyl groups also being optionally substituted with one or more substituents chosen from hydroxyl groups and halogen atoms or Si.

19. The aerosol device as claimed in claim 18, characterized in that the hydrophobic monomer is chosen from:
isoprene and butadiene;
methyl, ethyl, isobutyl, n-butyl, tert-butyl, ethylhexyl, furfuryl, isobornyl, tert-butylcyclohexyl or tert-butylbenzyl acrylate;
methyl, ethyl, n-butyl, isobutyl, hexyl or ethylhexyl methacrylate;
N—(C₆₋₁₂ alkyl)(meth)acrylamides;
the vinyl esters of formula: R₃—CO—O—CH═CH₂ in which R₃ represents a linear or branched alkyl group of 6 to 12 carbon atoms;
styrene;
vinyl acetate and vinylcyclohexane.

20. The aerosol device as claimed in any one of the preceding claims, characterized in that the composition-gradient copolymer(s) is or are present in an amount ranging from 0.1% to 20% by weight and preferably from 1% to 17% by weight relative to the total weight of the hair treatment composition and of the propellant.

21. The aerosol device as claimed in any one of the preceding claims, characterized in that the propellant essentially comprising dimethyl ether is present in an amount ranging from 20% to 60% by weight and preferably from 25% to 55% by weight relative to the total weight of the hair treatment composition and of the propellant.

22. The aerosol device as claimed in any one of the preceding claims, characterized in that the composition-gradient copolymer is present in dissolved form, or alternatively in the form of an aqueous or organic dispersion.

23. The aerosol device as claimed in any one of the preceding claims, characterized in that the cosmetically acceptable aqueous medium comprises water and/or one or more cosmetically acceptable solvents.

24. The aerosol device as claimed in claim 23, characterized in that the cosmetically acceptable solvents(s) is or are chosen from C₁-C₄ lower alcohols, polyols, polyol ethers and acetone, and mixtures thereof.

25. The aerosol device as claimed in claim 24, characterized in that the cosmetically acceptable solvent(s) is or are chosen from C₁-C₄ lower alcohols.

26. The aerosol device as claimed in claim 24 or 25, characterized in that the proportion of alcohol is between 1% and 70% by weight relative to the total weight of the hair treatment composition and of the propellant.

27. The aerosol device as claimed in any one of the preceding claims, characterized in that the water is present in an amount of between 20% and 95% by weight relative to the total weight of the hair treatment composition and of the propellant.

28. The aerosol device as claimed in any one of the preceding claims, characterized in that the hair treatment composition also comprises at least one adjuvant chosen from silicones in soluble, dispersed or microdispersed form, nonionic, anionic, cationic and amphoteric surfactants, ceramides and pseudoceramides, vitamins and provitamins including panthenol, plant, animal, mineral and synthetic oils, waxes, ceramides and pseudoceramides, water-soluble and liposoluble, silicone or nonsilicone sunscreens, mineral and organic, colored or uncolored pigments, dyes, nacreous agents and opacifiers, sequestrants, plasticizers, solubilizers, acidifying agents, basifying agents, mineral and organic thickeners, antioxidants, hydroxy acids, penetrants, fragrances and preserving agents.

29. The use of the product vaporized by the aerosol device as claimed in any one of claims 1 to 28, for shaping and/or holding the hairstyle.

30. A styling process comprising the step that consists in vaporizing the hair treatment composition contained in the aerosol device as claimed in any one of claims 1 to 28, onto wet or dry hair.

31. The use of at least one composition-gradient copolymer comprising at least two different monomers, and having a mass polydispersity index (Ip) of less than or equal to 2.5, in the presence of at least one silicone, in aerosol devices containing a propellant essentially comprising dimethyl ether.

32. The use as claimed in claim 31, characterized in that the propellant essentially comprising dimethyl ether comprises from 5% to 100% by weight and preferably from 20% to 60% by weight of dimethyl ether relative to the total weight of the propellant.

33. The use as claimed in claim 32, characterized in that the propellant other than dimethyl ether is chosen from hydrocarbons and compressed gases.

34. The use as claimed in claim 33, characterized in that the propellant other than dimethyl ether is chosen from C₁₋₅ alkanes, air, nitrogen and carbon dioxide.

35. The use as claimed in claim 33 or 34, characterized in that the propellant other than dimethyl ether is contained in an amount of from 0 to 95% by weight and preferably from 0 to 60% by weight relative to the total weight of the propellant.

36. The use as claimed in claim 33, 34 or 35, characterized in that the pressure of the compressed gas is between 2 and 14 bar.

37. The use as claimed in claim 31, characterized in that the propellant consists solely of dimethyl ether.

38. The use as claimed in any one of claims 31 to 37, characterized in that the mass polydispersity index (Ip) is between 1.1 and 2.3.

39. The use as claimed in any one of claims 31 to 38, characterized in that the silicone(s) is(are) chosen from organomodified or non-organomodified polydimethylsiloxanes.

40. The use as claimed in any one of claims 31 to 39, characterized in that the weight-average molecular mass of the composition-gradient copolymer is between 5000 and 1 000 000 g/mol.

41. The use as claimed in any one of claims 31 to 40, characterized in that the number-average molecular mass of the composition-gradient copolymer is between 5000 and 1 000 000 g/mol.

42. The use as claimed in any one of claims 31 to 41, characterized in that the composition-gradient copolymer is such that, on the adsorption chromatography (LAC) curve representing the proportion of polymers as a function of the elution volume, the difference (V^{1/2} max-V^{1/2} min) is less than or equal to 3.5 and preferably between 1 and 2.8, “V^{1/2} min” being the minimum value of the elution volume at the mid-height of the curve, and “V^{1/2} max” being the maximum value of the elution volume at the mid-height of the curve.

43. The use as claimed in any one of claims 31 to 42, characterized in that at least one of the monomers of the composition-gradient copolymer is a hydrophilic monomer.

44. The use as claimed in claim 43, characterized in that the hydrophilic monomer is chosen from:
C₁-C₄ aminoalkyl (meth)acrylate derivatives;
C₁-C₈ dialkylallylamines;
vinylamine;
vinylpyridines;
and also the mineral acid or organic acid salts thereof, or the quaternized forms thereof;
ethylenic carboxylic acids containing from 3 to 20 carbon atoms, or salts thereof;
carboxylic anhydrides bearing a vinyl double bond, containing from 4 to 30 carbon atoms;
ethylenic, sulfonic or phosphonic acids, and salts thereof;
vinyl alcohol;
acrylamide or methacrylamide, N—(C₁-C₆ alkyl)(meth)acrylamides, N,N-di(C₁-C₃ alkyl)(meth)acrylamides, N,N-di(C₁-C₄ alkyl)amino(C₁-C₆ alkyl)(meth)acrylamides;
hydroxy (C₂-C₄ alkyl) (meth)acrylates;
polyethylene glycol (5 to 100 ethylene oxide units) (meth)acrylates or glycol (meth)acrylates, which are unsubstituted or substituted on their end function with C₁₋₄ alkyl, phosphate, phosphonate or sulfonate groups;
C₁₋₄ alkoxyalkyl (meth)acrylates;
polysaccharide (meth)acrylates;
optionally cyclic vinylamides;
vinyl ethers;
methacrylamidopropoxytrimethylammonium;
N,N-dimethyl-N-methacryloxyethyl-N-(3-sulfopropyl)ammonium,
3-methacryloylethoxycarbonylpyridinium,
the compound of formula: 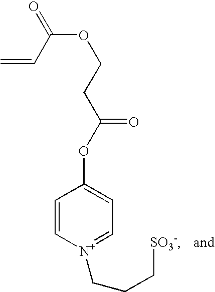
N-(3-sulfopropyl)-4-vinylpyridinium of formula:

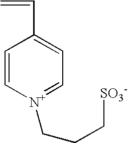

45. The use as claimed in claim 44, characterized in that the hydrophilic monomer is chosen from N,N-dimethylaminoethyl methacrylate (DMAEMA), acrylic acid, methacrylic acid, crotonic acid, styrenesulfonic acid, acrylamidopropanesulfonic acid, dimethylaminopropylmethacrylamide (DMAPMA); styrene-sulfonate, hydroxyethyl acrylate, glyceryl acrylate, methoxyethyl (meth)acrylate, ethoxyethyl (meth)acrylate, methoxypolyethylene glycol (8 or 12 EO) (meth)acrylate; hydroxypolyethylene glycol (meth)acrylate; N-vinyl-pyrrolidone, N-vinylcaprolactam, acrylamide and N,N-dimethyl-acrylamide.

46. The use as claimed in any one of claims 31 to 45, characterized in that at least one of the monomers of the composition-gradient copolymer is a hydrophobic monomer.

47. The use as claimed in claim 46, characterized in that the hydrophobic monomer is chosen from:
ethylenic hydrocarbons containing from 2 to 30 carbon atoms;
the acrylates of formula CH₂═CHCOOR₁, in which R₁ represents a saturated or unsaturated, linear, branched or cyclic hydrocarbon-based group containing from 1 to 30 carbon atoms, in which is(are) optionally intercalated one or more hetero atoms chosen from O, N, S and Si, said hydrocarbon-based group also possibly being substituted with one or more substituents chosen from hydroxyl groups and halogen atoms,
or R₁represents a group —(R₁₀)ₓ—(OC₂H₄)ₙ—OR₁₁, with x=0 or 1, R₁₀=saturated or unsaturated, linear or branched divalent hydrocarbon-based group containing from 1 to 30 carbon atoms, n=5 to 100 and R₁₁=H or CH₃;
the methacrylates of formula: CH₂═C(CH₃)—COOR₂, in which R₂ represents a saturated or unsaturated, linear, branched or cyclic hydrocarbon-based group containing from 1 to 30 carbon atoms, in which is(are) optionally intercalated one or more hetero atoms chosen from O, N, S and Si, said hydrocarbon-based group also being optionally substituted with one or more substituents chosen from hydroxyl groups and halogen atoms,
or R₂ represents —(R₁₀)ₓ—(OC₂H₄)ₙ—OR₁₁, with x=0 or 1, R₁₀=saturated or unsaturated, linear or branched divalent hydrocarbon-based group containing from 1 to 30 carbon atoms, n=5 to 100 and R₁₁=H or CH₃;
N—(C₈₋₃₀ alkyl)(meth)acrylamides;
the vinyl esters of formula: R₃—CO—O—CH═CH₂ in which R₃ represents a linear or branched alkyl group of 2 to 12 carbon atoms;
the vinyl compounds of formula: CH₂═CH—R₄ in which R₄ is a group —OC(O)—CH₃, a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; said cycloalkyl, aryl, aralkyl and heterocyclic groups being optionally substituted with one or more substituents chosen from hydroxyl groups, halogen atoms and linear or branched alkyl groups of 1 to 4 carbon atoms, in which is(are) optionally intercalated one or more hetero atoms chosen from O, N, S and P, and said alkyl groups also being optionally substituted with one or more substituents chosen from hydroxyl groups and halogen atoms or Si.

48. The use as claimed in claim 47, characterized in that the hydrophobic monomer is chosen from:
isoprene and butadiene;
methyl, ethyl, isobutyl, n-butyl, tert-butyl, ethylhexyl, furfuryl, isobornyl, tert-butylcyclohexyl or tert-butylbenzyl acrylate;
methyl, ethyl, n-butyl, isobutyl, hexyl or ethylhexyl methacrylate;
N—(C₆₋₁₂ alkyl)(meth)acrylamides;
the vinyl esters of formula: R₃—CO—O—CH═CH₂ in which R₃ represents a linear or branched alkyl group of 6 to 12 carbon atoms;
styrene;
vinyl acetate and vinylcyclohexane.

49. The use as claimed in any one of claims 31 to 48, characterized in that the composition-gradient copolymer is present in dissolved form, or alternatively in the form of an aqueous or organic dispersion.
